(19) United States
(12) Patent Application Publication
(10) Pub. No.: US 2010/0125330 A1
(43) Pub. Date: May 20, 2010

(54) SYNTHETIC VASCULAR PROSTHESIS AND METHOD OF PREPARATION

(76) Inventors: Bronislava G. Belenkaya, Campbell, CA (US); Edward C. Kwok, Cupertino, CA (US)

Correspondence Address: Bronislava G. Belenkaya, 350 Budd Avenue, Q9, Campbell, CA, 95008, US

(21) Appl. No.: 12/620,403

(22) Filed: Nov. 17, 2009

Related U.S. Application Data
(60) Provisional application No. 61/115,208, filed on Nov. 17, 2008.

(51) Int. Cl. A61F 2/82; B05D 7/00

(52) U.S. Cl. 623/1.46; 427/2.25

(57) ABSTRACT

A biocompatible small-diameter vascular graft, blood vessels conduit, or cell growth stimulator carrier composition which includes a completely biodegradable, hydrophilic non-gel material that has a controllable blood absorption or other biological liquid absorption ability, a controllable fiber architecture and pore sizes, and other biologically active properties, such as cell adhesion, proliferation and spreading, haemostatic and vascular tissue growth acceleration. The material retains its contour and shape when wet, and does not exhibit any swelling.

### CROSS REFERENCE TO RELATED APPLICATIONS

The present application is related to and claims priority to U.S. Provisional Patent Application, entitled “Synthetic Vascular Prosthesis and Method of Preparation,” Ser. No. 61/115,208, filed on Nov. 17, 2008. The Provisional patent application is hereby incorporated by reference in its entirety.

### BACKGROUND OF THE INVENTION

The present invention relates to biodegradable prosthetic vascular grafts. Specifically, the present invention relates to biodegradable synthetic vascular graft based on hydrophilic nonwoven scaffolds and devices thereof.

Vascular diseases are often treated by arterial bypass operations using autogenous grafts. For example, coronary artery bypass operations treat angina in life-threatening cardiovascular disease. These operations are costly and have significant mortality. Many patients do not have suitable veins (usually internal mammary artery or suphenous vein), there is a compliance mismatch between veins and arteries, and thrombosis and infection are also serious problems. Thus, there is strong demand for alternative vascular prosthesis based on biomaterials.

Various synthetic materials are readily available and are commonly used in engineered vascular grafts. Most of these materials, though inert, are regarded as foreign by the body and this limits their continuous suitability over time. The synthetic materials most used are expanded polytetrafluoromethylene (ePTFE) and knitted poly(ethylene terephthalate) (Dacron). After implantation, fibrous tissue may encapsulate the outside of these grafts and can grow into their microporous surfaces. The inner surface of the grafts becomes covered with a “pseudointima,” which consists of fibrin and fibroblasts. Because the materials are markedly hydrophobic and usually do not contain basic or acidic groups, these materials are not optimal for endothelial cell attachment, and ‘pseudointima’ is essentially the blood-biomaterial interface. In addition, unmodified e-PTFE is hydrophobic with a low surface energy and weak electrical charge that are not hospitable for endothelial cells.

Although grafts made from these synthetic biomaterials perform well when used to replace larger blood vessels, they are inadequate for replacing small diameter (e.g., <4 mm) vessel because of a tendency for thrombus induction, embolism and occlusion of the graft lumen, lack of compliance, and excessive intimal hyperplasia at anastomotic joints, and are prone to infection. Modifications of PTFE using physical and chemical methods have improved cell adhesion only in the short term. Moreover, synthetic grafts do not allow vessel remodeling or vascular physiological responsiveness. Such characteristics are of special value for adolescent and child patients. For these patients, reoperation procedures are often requires.

Biodegradable grafts based on hydrophobic PLA or PLGA Biofelt™ (from Concordia, Corp.) or tubular meshes (e.g., from Concordia, Corp.) are used for arterial and vein prosthesis, including tubular knittied PLA grafts coated by PLA-Poly(caprolactone) preceded by bone marrow cells. Although these materials may be replaced over time by vascular tissue, the blood-new vessel interface is poorly populated by endothelial cells, leading to hyperplasia and thrombus formation, as occurs in nondegradable biomaterials.

To stimulate endothelial cells attachment, chemical modification of micro/nanofibered tubular graft may be achieved by chemically bonding cell adhesion proteins (see, e.g., International Patent Application Publication WO 2007/090102 A2) to the surface of the fibers. The resulting material, however, is still hydrophobic, which leads to limited cells penetration, attraction and attachment.

Russian Patent No. 2031661 discloses a microfibrous wound-healing remedy which is used for first and outdoor aid. The material in the wound-healing remedy is prepared by a electrohydrodynamic method. The remedy includes poly-d,l-lactide, poly (N-vinyl)pyrrolidone and a powdered sorptive material, such as polysaccharides networks, polyacrylates, cellulose esters or polyvinyl alcohol derivatives. The material can absorb 5-8 g/g water or blood; exhibited haemostatic abilities within 40 seconds of application and achieves moderate wound healing effects. However, introduction of nondegradable or slowly degradable components such as polyvinyl alcohol derivatives into this material significantly decreases its biodegradation ability and limits its use for external applications.

Russian Parent 2120306 discloses a completely biodegradable two layer dressing for wounds and burns. The dressing consists of a baking thin film layer (25-30 μm) prepared from co-poly (lactide-caprolactone) or co-poly (lactide-glycolide) with a lactide/caprolactone ratio or lactide/glycolide ratio of at least 50% by weight and a wound-facing microfiber absorbent layer that includes comprising a polylactide and poly (N-vinyl)pyrrolidone blend with a ratio of polylactide/poly (N-vinyl)pyrrolidone from 90/10 to 70/30 w/w. The microfiber absorbent layer is deposited on the film by an electrohydrodynamic method. The wound-facing microfiber layer may also contain antiseptic or analgesic drugs and proteolytic ferments. The dressings can absorb water or biological liquids, including blood, at least at 12 g/g and biodegrade within 12-36 days. However, vapor penetration of such dressings is at most 3.1 mg/cm² hour which precludes their use as dressings for wounds and burns that exhibit intensive “breathing” (e.g., large external fresh burns, bleeding wounds or different kinds of external injuries). Furthermore these dressings have uncontrollable degradation times, which limit their applications to wound or burn treatments, especially to internal wound treatments.

U.S. Pat. No. 7,309,498 discloses a hydrophilic micro/nano fibered biodegradable absorbent with regulated hydrophilicity, which is based on a PLGA or PLA blend with Poly (N-vinyl)pyrrolidone or other Poly(N-vinyl) lactams prepared by an electrospinning process. The material may absorb up to 15 g/g of blood or other biological liquids, depending on the component of the blend ratio, has a predictable and controllable degradation time, and may contain protein or small molecular drugs, or hydroxyacids. The micro/nanofibered material may contain living cells or some hydrogels to mimic natural extracellular matrix (ECM). The material is applied to external/internal wound surface as a flat sheet. The material may have hemostatic action and accelerates wound healing process.

### SUMMARY OF THE INVENTION

One embodiment of the invention provides a biocompatible small-diameter vascular graft, blood vessels conduit, or cell growth stimulator carrier composition which includes a completely biodegradable, hydrophilic non-gel material that has a controllable blood absorption or other biological liquid absorption ability, a controllable fiber architecture and pore sizes, and other biologically active properties, such as cell adhesion, proliferation and spreading, haemostatic and vascular tissue growth acceleration. The material retains its contour and shape when wet, and does not exhibit any swelling.

One embodiment provides a completely biodegradable, hydrophilic microfiber/nano fibered matrix on the base of a blend of one or more synthetic biodegradable polyesters and one or more poly (N-vinyl) lactams. Such a material can be used in a variety of products, such as small-diameter vascular grafts of different size and hydrophilic biodegradable coatings for biodegradable and biostable synthetic blood conduits. The material stimulates cell adhesion and proliferation with a predictable mechanical strength, hydrophilicity and controllable absorption of biological liquids including blood.

One embodiment provides a synthetic, completely biodegradable, hydrophilic microfiber/nano fibered matrix that includes natural ECM proteins, including cell adhesion proteins. The material may be prepared for vascular cells adhesion, growth and proliferation. The matrix may include living cells that are seeded onto the material surface just before implantation or are pre-grown onto the material surface.

One embodiment provides a method for preparing a completely biodegradable, hydrophilic microfiber/nano fibered matrix based, small-diameter vascular graft or blood vessel conduit.

Synthetic biodegradable polyesters useful for preparing a completely biodegradable hydrophilic microfiber/nano fibered matrix of the invention include homopolymers of L (−), D (+), d, l-lactide, glycolide, caprolactone, p-dioxanon or any mixture thereof, copolymers of L (−), D (+), d, l-lactide and glycolide, or caprolactone, or p-dioxanon, or polyoxyethylene glycols, or any mixture thereof, or copolymers of glycolide and caprolactone, or p-dioxanon. or any mixture thereof or polytyrosine, or polyhydroxybutyrate.

The poly (N-vinyl) lactams useful in preparing an absorbent of the present invention includes homopolymers, copolymers of N-vinyl lactams such as N-vinylpyrrolidone, N-vinylbutyrolactam, N-vinylcaprolactam, and the like, prepared with minor amounts (e.g., up to about 20 weight percent) of one or more of other vinyl monomers that are capable of copolymerizing with the N-vinyl lactams like acrylic monomers or others. Of the poly (N-vinyl) lactam homopolymers, the poly (N-vinyl)pyrrolidone (PVP) homopolymers are preferred.

A completely biodegradable, hydrophilic microfiber/nano fibered matrix of the present invention may be prepared by an electrospinning process using a blend of poly (N-vinyl) lactam and a biodegradable polyester solution at a polyester/poly (N-vinyl) lactam ratio from about 99/1 to about 1/99 w/w, preferably from about 98/2 to about 50/50 w/w.

The present invention provides a completely biodegradable, hydrophilic microfiber/nano fibered matrix suitable for use in a small-diameter blood vessel or a blood conduit. The material is capable of absorbing a controllable amount of water or blood without swelling. The material is mechanically strong and has a predictable biodegradation time.

A completely biodegradable, hydrophilic microfiber/nano fibered matrix of the present invention suitable for use in small diameter blood vessels or blood conduits may include at least one additional ingredient, which may be a releasable or non releasable component of the matrix. Preferably, the releasable ingredient with controllable delivery may be any vascular growth factors, such as TGFβ1, PDGF-BB and VEGF. A non-releasable ingredient may be any natural ECM component, such as any cell adhesion proteins, proteoglycans, hyaluronic acids or peptides containing an amino acid sequence which stimulates cell adhesion (e.g., RGD). Such an ingredient accelerates cell adhesion and proliferation. These components need not be chemically bonded to the polymer micro/nano fibers, but may be physically bonded (e.g., by hydrogen bonding). Such a matrix may also be used as a transplantable solid support or a scaffold for living cells (e.g., for growing a blood vessel).

A completely biodegradable, hydrophilic microfiber/nano fibered matrix of the present invention suitable for use in a small-diameter blood vessel or a blood conduit can be prepared using electrospinning of a polymer blend solution. The electrospinning may be carried out at 20-120 kV (preferably, 20-40 kV) and at a gap distance 15-40 cm. The initial solution may contain a blend of a biodegradable polymer and a poly (N-vinyl) lactam and may also contain different natural ECM components.

The products based on the materials of the present invention have a good mechanical strength and preserve their shape under wet conditions.

The present invention is better understood upon consideration of the detailed description below, in conjunction with the accompanying drawings.

### BRIEF DESCRIPTION OF THE DRAWINGS

FIG. 1 shows an eletrospinning apparatus depositing micro/nanofibers onto a porous substrate prepared from hydrophobic polymers placed onto a flat surface.

FIG. 2 shows blood conduit 20 prepared from a completely biodegradable, hydrophilic microfiber/nano fibered ECM-like matrix, in accordance with one embodiment of the present invention.

### DETAILED DESCRIPTION

One embodiment of the invention provides a completely biodegradable, hydrophilic microfiber/nanofibered, ECM-like matrix that is suitable for use in grafting small-diameter blood vessels or blood conduits. The material property can be controlled to provide a predetermined degradation time, a selected hydrophilicity, and a controllable release of growth factors and other components suitable for cell attachment and proliferation. The goal is to provide a hospitable environment for host cells to regenerate and repair a damage blood vessels over a period of time, as the blood conduit graft biologically degrade and eventually disappears. The graft is preferably absorbent and retains biological fluid. The micro/nanofibered material maybe deposited on biodegradable or non biodegradable substrate and use as a stent-graft for supporting damaged and weaken blood vessel after angioplasty or other blood vessel treatment procedure. The biodegradable substrate may be made from PLA, PGA, Polytyrosine, Polyhydroxybutyrate, Polycaprolactone, Polycarbonate, Poly-p-dioxanone, their copolymers or mixtures thereof. and maybe porous or nonporous. Nondegradable substrate may be made of metals that are well known in the art for stents preparation, including nitinol. The micro/nanofibered material may contain drugs for sustained release to suppress inflammatory response or cell ingrowth into the lumen to avoid restenosis or vessel occlusion. Suitable drugs include Sirrolimus, Everolimus, Dexamethasone or mixture thereof.

In one embodiment, a material for the small-diameter blood vessels or blood conduits may be created from a two-component blend. One component may be a synthetic, biodegradable polyester selected to provide a desired biodegradation time. The biodegradable polyester may be selected from homopolymers or copolymers of L (−), D (+), d, l-lactide with glycolide, caprolactone, p-dioxanon, mixtures thereof, homopolymers or copolymers of caprolactone with L (−), D (+), d, l-lactide, glycolide, p-dioxanon and mixtures thereof, and copolymers of L (−), D (+), d, l-lactide, caprolactone, p-dioxanon with polyoxyethylene glycols (PEG) and mixtures thereof or polytyrosine, or polyhydroxybutyrate. The other component may be a poly (N-vinyl) lactam selected from homopolymers, copolymers of N-vinyl lactams such as N-vinylpyrrolidone, N-vinylbutyrolactam, N-vinylcaprolactam. In addition, the blend may further include minor amounts (e.g., up to about 10-15 weight percent) of one or more of other vinyl monomers copolymerizable with the N-vinyl lactams (e.g., acrylic acid, acryl amides or hydroxyalkylacrylates. Of the poly (N-vinyl) lactam homopolymers, the poly (N-vinyl)pyrrolidone (PVP) homopolymers are preferred. A variety of poly (N-vinyl)pyrrolidones are commercially available.

To prepare a material with a controlled hydrophilicity, the ratio of polyester/poly (N-vinyl) lactam is used in the range from about 99/1 to about 1/99, preferably from about 98/2 to about 50/50 w/w for polylactide, or co (poly-lactide-glycolide) with a lactide/glycolide ratio from about 99/1 to about 50/50. Preferably, a poly (N-vinyl)pyrrolidone is used. Preferably, the molecular weights of the two components are in the range from 3×10⁴ to 5×10⁵ Dalton for polyester, and from 0.5×10⁴ to 4×10⁶ Dalton for poly (N-vinyl)pyrrolidone. The biodegradable polyester component may contain caprolactone homopolymers or caprolactone copolymers with lactide, or glycolide, or p-dioxanon, with a caprolactone/lactide ratio from about 50/50 to about 10/90 w/w and with a molecular weight of at least 1.5×10⁵ Dalton for the polyester component. The polyester/poly (N-vinyl)pyrrolidone ratio may be selected from about 90/10 to about 70/30 w/w. The biodegradable polyester component may contain copolymers of glycolide and p-dioxanon with a glycolide/p-dioxanon ratio from about 50/50 to about 1/99 w/w.

Using the formulation described above, one embodiment provides a completely biodegradable, hydrophilic nonwoven absorbent that may consist of microfibers between 0.3-4 μm, is irreversible with non-leachable poly (N-vinyl)pyrrolidone (PVP). The material is found capable of swelling-free absorption at least 20 w/w in water or blood and/or other biological liquids, without changing the contour or shape of the device. The material is believed capable of delivering growth factors, cytokines and chemokines to a blood stream and contains components of natural ECM, such as hyaluronic acid, proteoglycans, proteins or small peptides comprising cell adhesion amino acids sequence. The material may be used in blood vessel tissue regeneration applications and may be applied as tubular graft that may be attached to a parent vessel by biodegradable sutures anastomosis or be immobilized onto biodegradable hydrophobic scaffolds, such as a polylactide mesh, a felt, a knitted tubular graft or a non-degradable scaffold to increase hydrophilicity and induce cells attachment and proliferation. The material and its degradation products are biocompatible. A product based on a material of the present invention has good mechanical strength and preserves its shape under wet conditions. Such a product can be sterilized by X-ray radiation. The material may be made soft and compliant with a parent blood vessel.

To obtain a completely biodegradable, hydrophilic unwoven absorbent, an electro-hydrodynamic method for solution spinning can be applied. Such a method involves spraying a solution of a polymer blend through a capillary nozzle onto a substrate. More particularly, the method may include a stream of compressed air or another gas through a capillary nozzle, and continuously introducing into the air stream a solution of a blend of a biodegradable polyester and poly (N-vinyl)pyrrolidone in a solvent (e.g. dichloromethane or mixture of ethyl acetate and a lower alcohol with the weight ratio of (5-10):1. Alternatively, the solution may be provided by a syringe without compressed air or gas. An exemplary concentration of the polymer in the solution is 1-40% w. The voltage between the nozzle and the substrate may be 20-120 kV. The negative pole is set at the metal capillary of the nozzle. The substrate is grounded. The gap between the nozzle and the substrate may be 15-30 cm. Depending on the voltage, the gap value, the speed of the compressed air stream, and solution concentration, a material of a controlled density, with a microfiber diameter that varies from 0.1-5 μm can be prepared. A microfiber unwoven tubular shaped material can be prepared using a cylindrical substrate. After the process, the material removed from the substrate may be vacuum dried. A finished product is packed and sterilized by γ-radiation using a conventional technique. Other electrospinning techniques may also be used.

The substrate may placed on a rotating drum or a mandrel, such as described in Russian patent RU 2121036 (20 Oct. 1998). Non-drum substrates, including non-moving substrates, can also be used. In one embodiment, the substrate is a hydrophobic porous material that can also be prepared by the electrospinning method described above. In that embodiment, the hydrophobic porous material consists of a biodegradable polymer.

Materials with different densities and mechanical strengths appropriate to withstand the blood pressure in a blood vessel prosthesis can be obtained by: 1) selecting the microfiber thickness and packing density; and 2) depositing using an electro-hydrodynamic microfiber depositing process onto tubular or flat sheet substrate prepared from a biodegradable or biostable material. By successively varying the solution composition, density and thickness deposited in each layer, the electro-hydrodynamic microfiber depositing process can provide a material with multiple layers of microfibers of different thicknesses and densities.

The blood vessel prosthesis may be prepared directly from depositing of micro/nanofibers onto a mandrel of corresponding diameter, or formed from a flat sheet by rolling it into tube and sealing using an initial polyester solution that does not include PVP. To prepare a material capable to sustain blood pressure at least 300-400 mm Hg, the microfiber packing density should be at least 8-9 mg/cm² with a fiber diameter up to 2 μm.

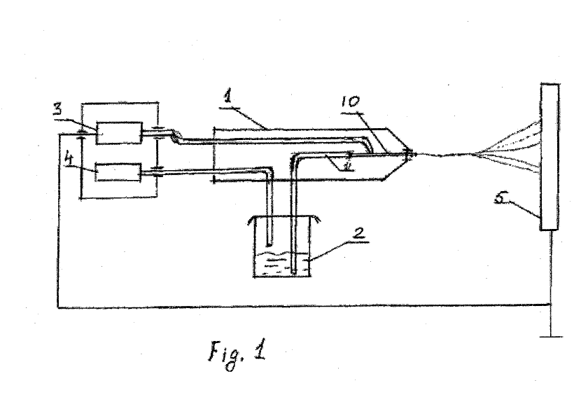

The micro/nanofibers maybe deposited onto a porous substrate prepared from hydrophobic polymers placed onto a mandrel or onto flat surface (FIG. 1). As shown in FIG. 1, electrospinning apparatus 1 deposits micro/nanofibers onto a porous substrate 5 prepared from hydrophobic polymers placed onto flat surface. Electrospinning apparatus 1 mixes compressed gas under pressure from gas source 3 at nozzle 10 with a stream of polymer solution supplied from source 2. Source 2 contains a blend of polymer dissolved in a selected solvent. The flow of the polymer solution may be controlled by pressure from gas source 4. Micro/nanofibers may be deposited on a tubular substrate using a mandrel or drum, which may be rotated for a uniform coating on the cylindrical surface. The fibers may be deposited on both sides or on one side of the substrate. To increase microfibers penetration into pores of the substrate, the fiber density should be at least 2.5 mg/cm².

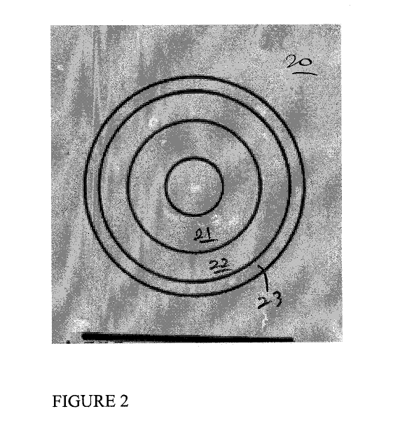

To provide a tubular blood vessel with an improved mechanical strength and a cell attachment ability based on a completely biodegradable, hydrophilic microfiber/nano fibered ECM-like matrix, the material may contain two or more microfiber layers of different compositions, different fiber sizes, and different packing densities. FIG. 2 shows blood conduit 20 prepared from a completely biodegradable, hydrophilic microfiber/nano fibered ECM-like matrix, in accordance with one embodiment of the present invention. As shown in FIG. 2, blood conduit 20 includes layers that mimicked a natural blood vessel: a) innermost layer 21, mimicking ‘tunica intima,’ may be formed by a hydrophilic layer of PLA, PLGA, PLC or a mixture thereof, and PVP; middle layer 22, mimicking ‘tunica media,’ may be a core layer providing the prosthesis mechanical strength, formed by a hydrophobic or weakly hydrophilic blend with Polyester/PVP<95/5 w/w prepared from a high molecular weight polyester (e.g., Poly-L,L-lactide, or Poly-Caprolactone, or copolymer with a high fiber packing density); and c) outmost layer 23, mimicking ‘tunica adventitia,’ may be a hydrophilic layer of PLA, PLGA, PLC or a mixture thereof and PVP. The hydrophilic layers are intended to allow cell migration from surrounding tissue; the hydrophobic Poly-d.l-lactide layer may prevent “vessel adhesion” to the surrounding tissue. Each layer may include different components of natural blood vessel ECM. Each layer may include at least one additional bioactive ingredient of natural blood vessel ECM, which may be releasable from the matrix and which may be immobilized into a polymeric matrix (e.g., by an electrospinning method). Alternatively, the innermost layer may be a hydrophobic layer with a smooth surface that is formed by polyester of a high molecular weight to prevent adhesion of blood cells or platelets which may cause blockage or clot.

A completely biodegradable, hydrophilic microfiber/nano fibered matrix suitable for use in a small-diameter blood vessel or a blood conduit may include at least one additional ingredient, which may be a releasable or a non-releasable component of the matrix. Preferably, the releasable ingredient with a controllable delivery may be a vascular endothelial growth factor (e.g., TGFβ1, PDGF-BB or VEGF). The non-releasable ingredient may be a natural ECM component, such as a cell adhesion protein, a proteoglycan, a hyaluronic acid or a peptide containing an amino acid sequence that stimulates cell adhesion (e.g., RGD). Such a material is believed to accelerate cell adhesion and proliferation. The components need not be chemically bonded to the polymer micro/nano fibers, but may be physically bonded (e.g., via hydrogen bonding). Such a matrix may also be used as a transplantable solid support or a scaffold for living cells (e.g., a living cell transplant for blood vessels).

When used to provide a prolonged and controlled drug release to a surface of an internal or external wound or burn, the material of the present invention may contain two or more microfiber layers of different compositions. Each layer may include a blend of the biodegradable polymer and poly (N-vinyl)pyrrolidone. Different layers may have different ratios of biodegradable polymer/poly (N-vinyl)pyrrolidone or different biodegradable polymers. Different types of polymers and/or copolymers may be used that may have different molecular weights, contain different biocompatible functional groups (e.g., hydroxyl, carboxyl, or amino groups) or contain different additives such as low or high molecular weight alcohols (e.g., sorbitol, mannitol, starch, polyoxyethylene glycols). Each layer may include at least one additional bioactive ingredient, which may be releasable from the absorbent and which may be immobilized into a polymeric matrix (e.g., by an electro-hydrodynamic method).

For use in a drug delivery system, the material of the present invention may contain drugs immobilized by an electro-hydrodynamic or another method, and then ground into fine particles of less than 10 μm in diameter. For drug carrier usage, the material may be prepared, for example, from a blend of polylactide and poly (N-vinyl)pyrrolidone, with a polylactide molecular weight of at least 5×10⁴ Dalton.

The above detailed description is provided to illustrate specific embodiments of the present invention and is not intended to be limiting. Numerous variations and modifications within the scope of the present invention are possible. The present invention is set forth in the accompanying claims.

We claim:

1. A completely biodegradable graft, comprising:
an outer absorbent layer comprising a blend of a biodegradable polymer and a lactam; and
an inner layer comprising a biodegradable polymer of a molecular weight greater than the biodegradable polymer of the outer absorbent layer.

2. A completely biodegradable graft as in claim 1, wherein the biodegradable polymer of the outer absorbent layer is selected from the group consisting of poly-lactic acid (PLA), co-polymer of poly-glycolic acid and lactic acid, poly-caprolatone (PLC) or a mixture thereof.

3. A completely biodegradable graft as in claim 1, wherein the lactam of the outer absorbent layer is selected from the group consisting of homopolymers, copolymers of N-vinyl lactams.

4. A completely biodegradable graft as in claim 3, wherein the N-vinyl lactams is selected from the group consisting of N-vinylpyrrolidone, N-vinylbutyrolactam and N-vinylcaprolactam.

5. A completely biodegradable graft as in claim 1, wherein the biodegradable polymer of the outer absorbent layer comprises a biodegradable polyester selected from the group consisting of homopolymers or copolymers of L (−), D (+), d, l-lactide with glycolide, caprolactone, p-dioxanon, and mixtures thereof, homopolymers or copolymers of caprolactone with L (−), D (+), d, l-lactide glycolide p-dioxanon and mixtures thereof, and copolymers of L (−), D (+), d, l-lactide, caprolactone, p-dioxanon with polyoxyethylene glycols (PEG) and mixtures thereof, polytyrosines, and polyhydroxybutyrates.

6. A completely biodegradable graft as in claim 1, wherein the inner layer is hydrophobic or weakly absorbent.

7. A completely biodegradable graft as in claim 1, wherein the inner layer comprises a blend of a polyester and a lactam in the ratio of less than 95/5 w/w.

8. A tubular completely biodegradable graft as in claim 1, further comprising an inner absorbent layer being provided inside of the inner layer.

9. A completely biodegradable graft as in claim 1, further comprising a releasable component

10. A completely biodegradable graft as in claim 9, wherein the releasable component comprises a vascular growth factor, cytokines or chemokines.

11. A completely biodegradable graft as in claim 10, wherein the growth factor is selected from the group consisting of: TGFβ1, PDGF-BB and VEGF.

12. A completely biodegradable graft as in claim 1, further comprising a non-releasable component.

13. A completely biodegradable graft as in claim 12, wherein the non-releasable component is selected from the group consisting of: cell adhesion protein, a proteoglycan, a hyaluronic acid and a peptide containing an amino acid sequence that stimulates cell adhesion.

14. A completely biodegradable graft as in claim 13, wherein the peptide comprises RGD.

15. A completely biodegradable graft as in claim 1, wherein the outer absorbent layer is seeded with living cells.

16. A completely biodegradable graft as in claim 1, further comprising a plurality of additional absorbent layers each comprising a blend of a biodegradable polymer and a lactam.

17. A completely biodegradable graft as in claim 16, wherein different layers of the additional absorbent layers polymers of different molecular weights.

18. A completely biodegradable graft as in claim 16, wherein the biodegradable polymers of different ones of the additional absorbent layers contain different biocompatible functional groups or alcohols of different molecular weights.

19. A completely biodegradable graft as in claim 18, wherein the functional groups are selected from the group consisting of hydroxyl, carboxyl, and amino groups,

20. A completely biodegradable graft as in claim 19, wherein the alcohols are selected from the group consisting of sorbitol, mannitol, starch, and polyoxyethylene glycols.

21. A completely biodegradable graft as in claim 1, wherein the completely biodegradable graft is tubular.

23. A completely biodegradable graft as in claim 1, wherein a drug is immobilized in the completely biodegradable graft.

24. A completely biodegradable graft as in claim 1, further comprising one or more vinyl monomers copolymerizable with the N-vinyl lactams.

25. A completely biodegradable graft as in claim 24, wherein the vinyl monomers are selected from the group consisting of acrylic acid, acryl amides and hydroxyalkylacrylates.

26. A completely biodegradable graft as in claim 1, wherein the outer absorbent layer comprises microfibers between 0.3-4 μm.

27. A completely biodegradable graft as in claim 1, wherein the microfibers are electro-hydrodynamic deposited

28. A completely biodegradable graft as in claim 27, wherein the microfiber packing density is at least 8-9 mg/cm²

29. A completely biodegradable graft as in claim 1, wherein the biodegradable graft is mechanically resilient to withstand a pressure of 300-400 mm Hg.

30. A method for preparing a completely biodegradable graft, comprising:
electro-hydrodynamically spraying a first solution through a capillary nozzle onto a substrate to form a first layer of microfibers; and
electro-hydrodynamically spraying a second solution onto the first layer of microfibers that is formed on the substrate to form a second layer of microfibers, wherein the second solution comprises a blend of a biodegradable polymer and a lactam provided in a solvent.

31. A method as in claim 31, wherein a voltage of 20-120 KV is imposed between the nozzle and the substrate, at a distance between 15 cm and 30 cm.

32. A method as in claim 31, further comprising:
removing the substrate with the first and second layers formed thereon; and
vacuum drying the removed substrate.

33. A method as in claim 31, wherein the substrate is placed on a grounded flat surface.

34. A method as in claim 31, wherein the substrate is placed on a grounded tubular surface.

35. A method as in claim 31, wherein the biodegradable polymer of the second solution is selected from the group consisting of poly-lactic acid (PLA), co-polymer of poly-glycolic acid and lactic acid, poly-caprolatone (PLC) or a mixture thereof.

36. A method as in claim 31, wherein the lactam of the second solution is selected from the group consisting of homopolymers, copolymers of N-vinyl lactams.

37. A method as in claim 36, wherein the N-vinyl lactams is selected from the group consisting of N-vinylpyrrolidone, N-vinylbutyrolactam and N-vinylcaprolactam.

38. A method as in claim 31, wherein the biodegradable polymer of the second solution comprises a biodegradable polyester selected from the group consisting of homopolymers or copolymers of L (−), D (+), d, l-lactide with glycolide, caprolactone, p-dioxanon, and mixtures thereof, homopolymers or copolymers of caprolactone with L (−), D (+), d, l-lactide glycolide p-dioxanon and mixtures thereof, and copolymers of L (−), D (+), d, l-lactide, caprolactone, p-dioxanon with polyoxyethylene glycols (PEG) and mixtures thereof, polytyrosines, and polyhydroxybutyrates.
